(19) United States
(12) Patent Application Publication
(10) Pub. No.: US 2017/0007210 A1
(43) Pub. Date: Jan. 12, 2017

(54) BLOOD VESSEL DIAMETER MEASURING APPARATUS

(71) Applicants: TOKYO UNIVERSITY OF AGRICULTURE AND TECHNOLOGY, Tokyo (JP); NIHON KOHDEN CORPORATION, Tokyo (JP)

(72) Inventors: Kohji MASUDA, Tokyo (JP); Shinya ONOGI, Tokyo (JP); Yoshinobu ONO, Tokyo (JP); Tsuneo TAKAYANAGI, Tokyo (JP); Takashi KAIAMI, Tokyo (JP)

(73) Assignees: TOKYO UNIVERSITY OF AGRICULTURE AND TECHNOLOGY, Tokyo (JP); NIHON KOHDEN CORPORATION, Tokyo (JP)

(21) Appl. No.: 15/198,033

(22) Filed: Jun. 30, 2016

(30) Foreign Application Priority Data
Jul. 8, 2015 (JP) 2015-136753

(51) Int. Cl. A61B 8/08; A61B 8/00; A61B 8/15

(52) U.S. Cl. CPC A61B 8/5223; A61B 8/0891; A61B 8/15; A61B 8/4254; A61B 8/4209; A61B 8/4218

(57) ABSTRACT

A blood vessel diameter measuring apparatus includes an ultrasonic probe which emits an ultrasonic wave toward a blood vessel, receives an ultrasonic wave reflected from the blood vessel and outputs an ultrasonic signal based on the received ultrasonic wave, a detecting section which is attached to the ultrasonic probe and outputs information indicating a position of the ultrasonic probe and an attitude of the ultrasonic probe with respect to the blood vessel, a support fixture which supports the ultrasonic probe so as to enable the ultrasonic probe to perform at least one of operations, an image processing section which produces an ultrasonic image of the blood vessel by using the ultrasonic signal from the ultrasonic probe and the position/attitude information from the detecting section, and a measuring section which measures a blood vessel diameter of the blood vessel by using the produced ultrasonic image.

### CROSS REFERENCE TO RELATED APPLICATION

This application is based on Japanese Patent Applications No. 2015-136753 filed on Jul. 8, 2015, the contents of which are incorporated herein by reference.

### BACKGROUND

The presently disclosed subject matter relates to a blood vessel diameter measuring apparatus which can accurately measure the blood vessel diameter of the artery irrespective of the skills of the operator.

Recently, researches showing that arteriosclerosis develops while showing occurrence of a failure of the vascular endothelial function as the initial phase have been conducted. In order to prevent arteriosclerosis, techniques and apparatuses for evaluating the vascular endothelial function have been developed. As a reliable technique for evaluating the vascular endothelial function, there is an apparatus called an FMD (Flow-Mediated Dilation) measurement system.

The apparatus measures the following phenomenon appearing in a blood vessel (hereinafter, it means the artery unless otherwise described), and evaluates the vascular endothelial function. That is, the blood flow in a blood vessel is stopped for a constant period of time (avascularization), and then the avascularization is released. At this time, in the case of a normal vessel, the production of NO which is a vasodepressor material from vascular endothelial cells is promoted by shear stress of the intima of the vessel due to a blood flow immediately after avascularization. As a result, after avascularization is released, the blood vessel diameter is gradually increased, and, after the blood vessel diameter reaches the maximum diameter, the blood vessel diameter is gradually decreased to return to that at rest. By contrast, in the case where a disorder exists in the vascular endothelial function, the maximum diameter when the vessel is expanded is smaller than that in the normal state (the degree of the expansion is smaller). When the change in blood vessel diameter before and after avascularization is measured, therefore, the vascular endothelial function can be evaluated.

Specifically, the measurement and the evaluation are performed in the following manner. A cuff which is same or similar to that for measuring the blood pressure is attached to the arm of the subject. Before the arm is pressurized by the cuff to perform avascularization, the blood vessel diameter upstream or downstream of the portion pressurized by the cuff is measured by using an ultrasonic echo system (the blood vessel diameter at rest). Thereafter, avascularization is performed for a constant period of time (about five minutes) at a pressure which is equal to or higher than the systolic blood pressure, and then the avascularization is released. Immediately after avascularization is released, the blood vessel diameter is continuously measured by the ultrasonic echo system, and the blood vessel diameter when the vessel is maximally expanded is obtained. The vascular endothelial function is evaluated based on a comparison between the blood vessel diameter at rest, and the blood vessel diameter when the vessel is maximally expanded after release of avascularization.

In order to correctly measure the blood vessel diameter of the subject, in the case where the measurement is performed in the short axis, an ultrasonic probe must be perpendicular to the blood vessel, and, in the case where the measurement is performed in the long axis, an ultrasonic probe must visualize the center of the blood vessel. In both the cases, the operator is required to have sophisticated skills. By contrast, in the technique disclosed in Japanese Patent No. 5,014,051, a special ultrasonic probe configured by two short-axis ultrasonic array probes and one long-axis ultrasonic array probe is used, and a dedicated control device is used, thereby simplifying measurement.

In the conventional methods, in the case where a usual ultrasonic probe is used, an inspection must be performed by an operator having sophisticated skills. In the case where, as disclosed in Patent Literature 1, a special ultrasonic probe for measuring the blood vessel diameter is used, and a dedicated control device is used, the device cannot be used in another ultrasonic inspection device.

Therefore, the presently disclosed subject matter provides a blood vessel diameter measuring apparatus which, irrespective of the skill of the operator and without using a dedicated device, can accurately measure the blood vessel diameter by using a usual ultrasonic probe.

### SUMMARY

The blood vessel diameter measuring apparatus of the presently disclosed subject matter has an ultrasonic probe, a position/attitude detecting section, a support fixture, an image processing section, and a measuring section.

The ultrasonic probe emits an ultrasonic wave toward a blood vessel, receives an ultrasonic wave reflected from the blood vessel, and outputs the received ultrasonic wave as an ultrasonic signal. The position/attitude detecting section is attached to the ultrasonic probe, and outputs position/attitude information indicating a position of the ultrasonic probe, and an attitude of the ultrasonic probe with respect to the blood vessel. The support fixture supports the ultrasonic probe so as to enable the ultrasonic probe to perform at least one of operations including: inclination in a direction which is inclined with respect to a longitudinal direction of the blood vessel; movement in the longitudinal direction of the blood vessel; and rotation from the longitudinal direction of the blood vessel to a direction which intersects with the longitudinal direction. The image processing section produces an ultrasonic image of the blood vessel by using the ultrasonic signal output from the ultrasonic probe, and the position/attitude information output from the position/attitude detecting section. The measuring section measures the blood vessel diameter of the blood vessel by using the produced ultrasonic image.

According to the blood vessel diameter measuring apparatus of the presently disclosed subject matter, an ultrasonic image of a blood vessel is produced by using the ultrasonic signal output from the ultrasonic probe which is supported by the support fixture, and the position/attitude information output from the position/attitude detecting section. The ultrasonic probe is not required to be completely perpendicular to the blood vessel. Therefore, the blood vessel diameter can be accurately measured irrespective of the skill of the operator who handles the ultrasonic probe, and without using a dedicated device.

### BRIEF DESCRIPTION OF DRAWINGS

FIG. 1 is a block diagram illustrating a blood vessel diameter measuring apparatus of an embodiment.

FIG. 2 illustrates an example of a support fixture in the embodiment.

FIG. 3 illustrates another example of the support fixture in the embodiment.

FIG. 4 is a block diagram illustrating an image processing section and measuring section in the embodiment.

FIG. 5 is a flowchart illustrating an operation of acquiring an ultrasonic image by the blood vessel diameter measuring apparatus of the embodiment.

FIG. 6 is a flowchart illustrating an operation of calculating the blood vessel diameter by the blood vessel diameter measuring apparatus of the embodiment.

FIG. 7 illustrates extraction of the vascular intima and calculation of the center of a blood vessel.

FIG. 8 illustrates calculation of the center line of a blood vessel.

FIG. 9 illustrates production of a pseudo long-axis tomographic image.

FIG. 10 is an operation flowchart illustrating a process in which the blood vessel diameter measured by the blood vessel diameter measuring apparatus of the embodiment is applied to an FMD test.

FIG. 11 illustrates a display example of a result of the FMD test.

### DETAILED DESCRIPTION OF EMBODIMENTS

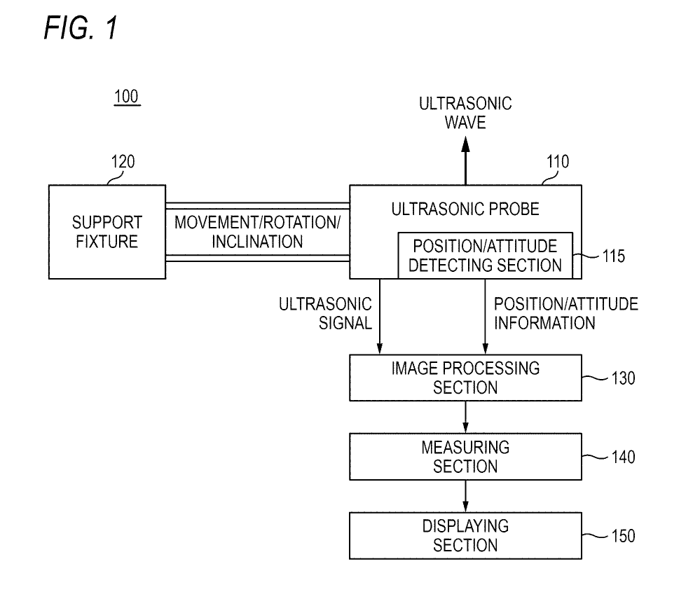

Next, the blood vessel diameter measuring apparatus of the presently disclosed subject matter will be described in detail with reference to the drawings. FIG. 1 is a block diagram of a blood vessel diameter measuring apparatus of an embodiment.

The blood vessel diameter measuring apparatus 100 of the embodiment has an ultrasonic probe 110, a position/attitude detecting section 115, a support fixture 120, an image processing section 130, a measuring section 140, and a displaying section 150.

The ultrasonic probe 110 emits an ultrasonic wave toward a blood vessel, receives an ultrasonic wave reflected from the blood vessel, and outputs the received ultrasonic wave as an ultrasonic signal. The ultrasonic signal is used for measuring the state of the blood vessel.

The position/attitude detecting section 115 is provided in the ultrasonic probe 110 in order to, when the ultrasonic probe 110 emits the ultrasonic wave toward the blood vessel, detect the position of the ultrasonic probe 110, and the attitude of the ultrasonic probe 110 with respect to the blood vessel. Specifically, the position/attitude detecting section 115 is configured by one of sensors which are usually used, such as a gyro sensor, an acceleration sensor, a motion sensor, an angular velocity sensor, a magnetic sensor, an infrared sensor, an encoder, and other non-contact sensor, or an arbitrary combination of these sensors. The position/attitude detecting section 115 is disposed in the ultrasonic probe 110 in order to know the position from which the ultrasonic probe 110 emits an ultrasonic wave to the blood vessel, and the angle at which the ultrasonic probe 110 emits the ultrasonic wave to the blood vessel.

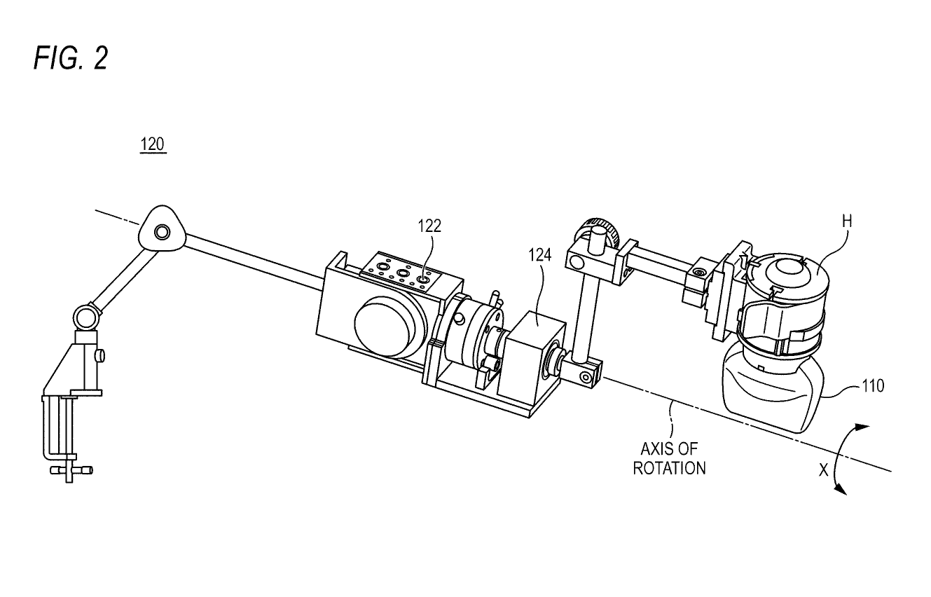

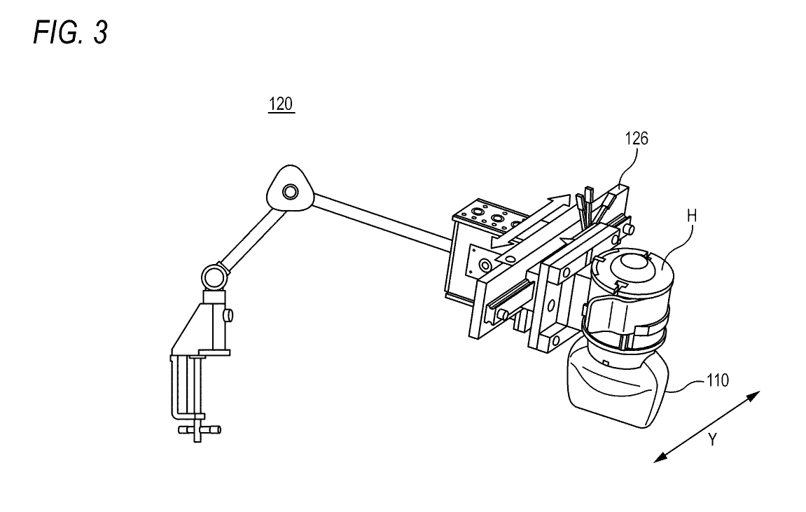

The support fixture 120 supports the ultrasonic probe 110 in a fixable manner, movable, and swingable manner. The support fixture 120 can perform on the ultrasonic probe 110, at least one of operations including: inclination in a direction which is inclined with respect to the longitudinal direction of the blood vessel; movement in the longitudinal direction of the blood vessel; and rotation from the longitudinal direction of the blood vessel to a direction which intersects with the longitudinal direction of the blood vessel. The support fixture performs such an operation in order to fix the ultrasonic probe 110 to a position suitable for measurement of the blood vessel diameter. The structure of the support fixture 120 will be specifically described with reference to FIGS. 2 and 3.

The support fixture 120 can locate the ultrasonic probe 110 in a place where the brachial artery of the subject can be visualized. During a period when the support fixture 120 moves the ultrasonic probe 110 to various positions and attitudes, the ultrasonic probe 110 outputs an ultrasonic signal in respective positions and attitudes. In response to the output, the position/attitude detecting section 115 outputs position/attitude information. The support fixture 120 and the ultrasonic probe 110 perform the above-described operation in both modes or before avascularization and after release of avascularization.

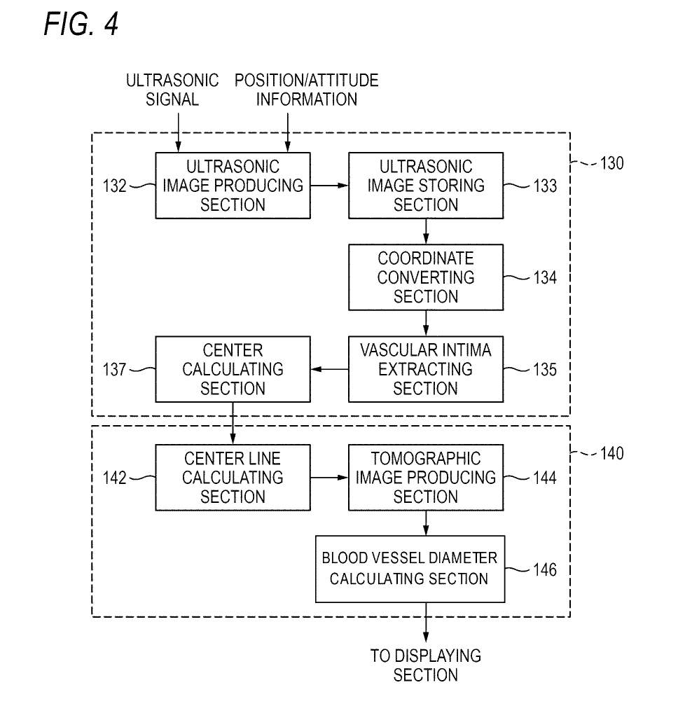

The image processing section 130 produces an ultrasonic image of the blood vessel by using the ultrasonic signal output from the ultrasonic probe 110, and the position/attitude information. The image processing section 130 produces an ultrasonic image of the blood vessel in both modes or before avascularization and after release of avascularization. The configuration of the image processing section 130 will be specifically described with reference to FIG. 4.

The measuring section 140 measures the blood vessel diameter of the blood vessel by using the produced ultrasonic image produced by the image processing section 130. The configuration of the measuring section 140 will be specifically described with reference to FIG. 4.

The displaying section 150 displays the ultrasonic image produced by the image processing section 130, and the blood vessel diameter measured by the measuring section 140. Specifically, the displaying section 150 is a fiat display device and/or a printer.

FIG. 2 is a structural view of an example of the support fixture in the embodiment. The support fixture 120 of FIG. 2 can incline the ultrasonic probe 110 in a direction which is inclined with respect to the longitudinal direction of the blood vessel. The support fixture 120 has a motor 122 for inclining the ultrasonic probe 110. When the motor 122 rotates, a rotation shaft of a holding member 124 is swung in the direction of the arrow X in FIG. 2. When the rotation shaft of the holding member 124 is swung, the ultrasonic probe 110 is inclined in a direction which is inclined with respect to the longitudinal direction of the blood vessel. The support fixture 120 can stably support the ultrasonic probe 110 at various inclination angles. Alternatively, the motor 122 may not be used, and the operator may manually fix the ultrasonic probe 110 in an arbitrary position.

FIG. 3 is a structural view of another example of the support fixture in the embodiment. The support fixture 120 of FIG. 3 can move the ultrasonic probe 110 in the longitudinal direction of the blood vessel. The support fixture 120 has a linear motor 126 for moving the ultrasonic probe 110. When the linear motor 126 operates, the ultrasonic probe 110 can be parallel moved in the direction of the arrow Y in FIG. 3. The support fixture 120 can stably support the ultrasonic probe 110 in various positions of the blood vessel. Alternatively, the linear motor 126 may not be used, and the operator may manually fix the ultrasonic probe 110 in an arbitrary position.

The support fixtures 120 of FIGS. 2 and 3 are not provided with a structure for rotating the ultrasonic probe 110 from the longitudinal direction of the blood vessel to a direction which intersects with the longitudinal direction. In order to enable the ultrasonic probe 110 to be rotated from the longitudinal direction of the blood vessel to a direction which intersects with the longitudinal direction, for example, a motor for rotating the ultrasonic probe 110 may be disposed in a fixing member H which fixes the ultrasonic probe 110 in FIGS. 2 and 3.

In stead of the configurations in each of which, as illustrated in FIGS. 2 and 3, the support fixture 120 separately includes the structure for inclining the ultrasonic probe 110 in a direction which is inclined with respect to the longitudinal direction of the blood vessel, or that for moving the ultrasonic probe 110 in the longitudinal direction of the blood vessel, or a structure which is not illustrated in FIGS. 2 and 3, and which rotates the ultrasonic probe 110 from the longitudinal direction of the blood vessel to a direction which intersects with the longitudinal direction, the support fixture 120 may include an appropriate combination of plural ones of the three structures.

FIG. 4 is a block diagram of the image processing section and measuring section in the embodiment. The image processing section 130 has an ultrasonic image producing section 132, an ultrasonic image storing section 133, a coordinate converting section 134, a vascular intima extracting section 135, and a center calculating section 137.

The ultrasonic image producing section 132 produces an ultrasonic image by using the ultrasonic signal output from the ultrasonic probe 110 (see FIG. 1). In the production of an ultrasonic image, a technique which is conventionally used is employed. The ultrasonic image producing section 132 adds the position/attitude information output from the position/attitude detecting section 115 (see FIG. 1) of the ultrasonic probe 110, to each of produced ultrasonic images in order to enable the direction in which each ultrasonic image is taken with respect to the blood vessel, to be known.

The plurality of ultrasonic images which are necessary for calculation of the blood vessel diameter are taken in both the modes, or before avascularization, i.e., at a timing before pressurization of the front arm of the subject by the cuff, and after release of avascularization, i.e., at that after pressurization of the front arm of the subject by the cuff. After release of avascularization, in order to obtain blood vessel diameter when the blood vessel is maximally expanded, a plurality of ultrasonic images are continuously taken. This enables the blood vessel diameters before pressurization and after release of avascularization to be compared with each other. In each of modes or before avascularization and after release of avascularization, a plurality of ultrasonic images are taken in different positions of the blood vessel, and in different attitudes of the ultrasonic probe 110 with respect to the blood vessel.

The ultrasonic image storing section 133 stores: the plurality of ultrasonic images which are produced by the ultrasonic image producing section 132, and in which, in both the modes or before avascularization and after release of avascularization, the positions and the attitudes are different; and the position/attitude information added to the ultrasonic images.

The coordinate converting section 134 coordinate-converts the attitudes of all the ultrasonic images stored in the ultrasonic image storing section 133, and interpolates the coordinate-converted images, thereby building a three-dimensional image. The coordinate converting section 134 has a reference coordinate system, and makes the ultrasonic images correspond to positions of the reference coordinates by using the position/attitude information added to the ultrasonic images, respectively. The coordinate converting section 134 can build a three-dimensional image of the blood vessel from ultrasonic images in various positions.

The vascular intima extracting section 135 extracts the vascular intima based on the images which are coordinate-converted by the coordinate converting section 134.

The center calculating section 137 performs the elliptic approximation on the shape of the vascular intima extracted by the vascular intima extracting section 135, and obtains the center of an ellipse formed by the elliptic data. The obtained center is the center of the blood vessel. The process of performing the elliptic approximation based on the shape of the vascular intima is performed on all vascular intimas extracted by the vascular intima extracting section 135. The following process may be performed in place of the elliptic approximation. Namely, a plurality of elliptic data may be previously stored, the center of an ellipse may be obtained based on stored elliptic data which substantially coincide with the shape of the extracted vascular intima, and the obtained center may be set as the center of the blood vessel.

The measuring section 140 has a center line calculating section 142, a tomographic image producing section 144, and a blood vessel diameter calculating section 146.

The center line calculating section 142 successively connects the centers of the blood vessel which are obtained by the center calculating section 137, to calculate the center line of the blood vessel.

The tomographic image producing section 144 rebuilds an image from the three-dimensional image so that the center line of the blood vessel which is calculated by the center line calculating section 142 is a straight line, and extracts cross sections which extend along the center line to produce a pseudo long-axis tomographic image.

The blood vessel diameter calculating section 146 obtains the blood vessel diameter by using the pseudo long-axis tomographic image produced by the tomographic image producing section 144. The obtained blood vessel diameter is output to the displaying section 150 (see FIG. 1).

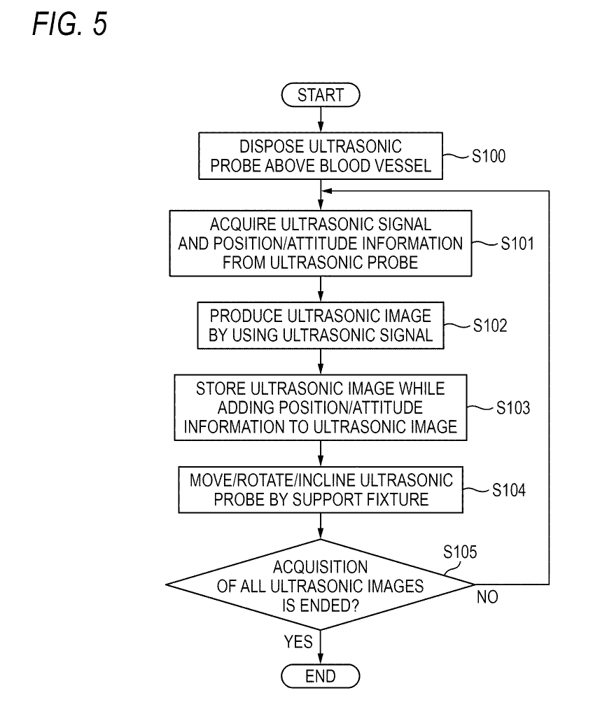

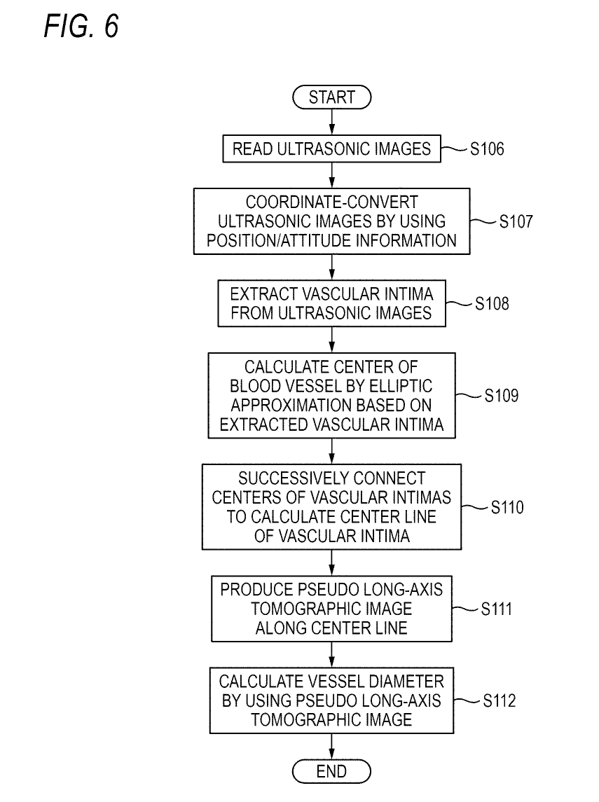

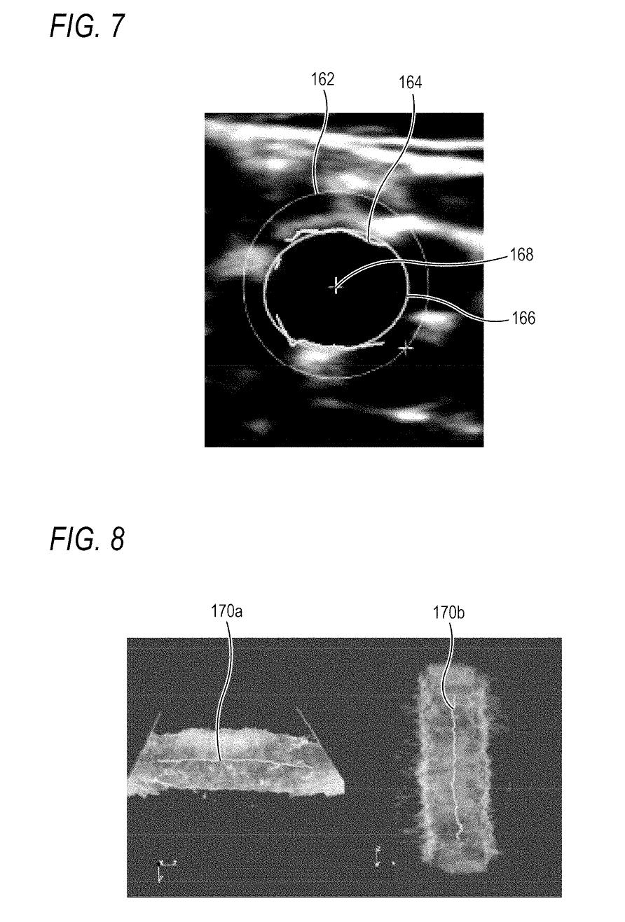

Next, the operation of the blood vessel diameter measuring apparatus of the embodiment will be described in detail with reference to the flowcharts of FIGS. 5 and 6. In the following description of FIGS. 5 and 6, the description will be made with reference to FIGS. 7 to 9.

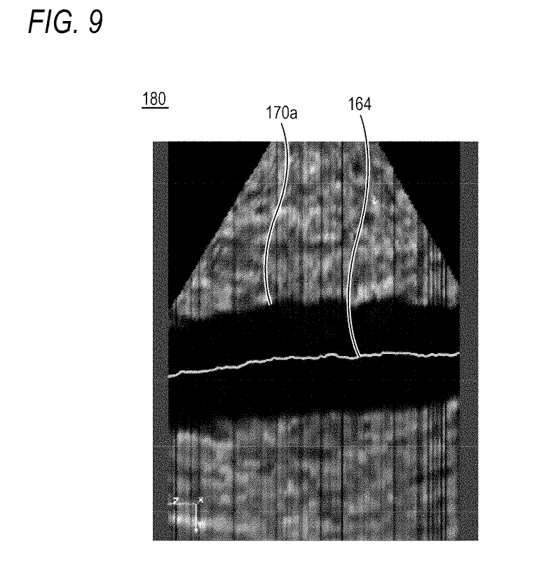

FIG. 5 is a flowchart of an operation of acquiring an ultrasonic image by the blood vessel diameter measuring apparatus of the embodiment. FIG. 6 is a flowchart of an operation of calculating the blood vessel diameter by the blood vessel diameter measuring apparatus of the embodiment, FIG. 7 illustrates extraction of the vascular intima and calculation of the center of the blood vessel, FIG. 8 illustrates calculation of the center line of the blood vessel, and FIG. 9 illustrates production of the pseudo long-axis tomographic image.

First, the operation of acquiring an ultrasonic image will be described with reference to the flowchart of FIG. 5. When the blood vessel diameter is to be measured, the operator places the ultrasonic probe 110 in a position where the brachial artery can be visualized from the surface of the upper arm of the subject, by using the support fixture 120 shown in FIG. 2 or 3 (step S100).

The ultrasonic image producing section 132 acquires the ultrasonic signal and position/attitude information from the ultrasonic probe 110. Specifically, the ultrasonic image producing section 132 acquires the ultrasonic signal output from the ultrasonic probe 110, and also the position/attitude information output from the position/attitude detecting section 115 of the ultrasonic probe 110 (step S101).

The ultrasonic image producing section 132 produces an ultrasonic image by using the acquired ultrasonic signal. The ultrasonic image is produced by a technique which is conventionally employed (step S102).

The ultrasonic image producing section 132 adds the position/attitude information to the produced ultrasonic image, and causes the resulting image to be stored in the ultrasonic image storing section 133 (step S103).

As a result of the above-described process of steps S100 to S103, one ultrasonic image is stored in the ultrasonic image storing section 133. Next, the ultrasonic probe 110 is moved, rotated, or inclined to a position which is different from the current position, by the motor 122 of the support fixture 120, and an ultrasonic image of the blood vessel in a different position and attitude is acquired (step S104).

The process of steps S100 to S104 are repeated until acquisition of ultrasonic images of the blood vessel in all positions and attitudes which are to be measured is ended (step S105: NO). If acquisition of all ultrasonic images is ended (step S105: YES), the operation of acquiring ultrasonic images is ended.

When ail ultrasonic images have been acquired as described above, the blood vessel diameter is then calculated by using the images. The operation of calculating the blood vessel diameter will be described with reference to the flowchart of FIG. 6. The calculation of the blood vessel diameter may be performed subsequently to the acquisition of ultrasonic images, or after elapse of a certain period of time. In the latter case, the ultrasonic images stored in the ultrasonic image storing section 133 may be fetched in an external apparatus such as a personal computer in which a blood vessel diameter calculating program is installed, and the blood vessel diameter may be calculated by the external apparatus.

The coordinate converting section 134 reads the plurality of ultrasonic images stored in the ultrasonic image storing section 133 (step S106). The coordinate converting section 134 coordinate-converts each of the ultrasonic images by using the position/attitude information added to the ultrasonic image (step S107).

The vascular intima extracting section 135 extracts the vascular intima from an ultrasonic image which is coordinate-converted by the coordinate converting section 134, and which is as shown in FIG. 7 (step S108). FIG. 7 illustrates extraction of the vascular intima and calculation of the center of the blood vessel. When the coordinate converting section 134 coordinate-converts the ultrasonic images stored in the ultrasonic image storing section 133, a sectional image of the blood vessel is obtained as shown in FIG. 7.

There is a possibility that sectional images of a plurality of blood vessels (arteries and veins) exist in the ultrasonic image of FIG. 7. Therefore, the operator previously designates the target blood vessel by a circle 162. The extraction of the vascular intima 164 is performed in the circle 162 by a technique such as edge detection which is usually used in image processing.

The center calculating section 137 performs the elliptic approximation on the shape of the vascular intima 164 extracted by the vascular intima extracting section 135, and calculates elliptic data. The center calculating section 137 obtains the center 168 of an ellipse formed by the calculated elliptic data 166 (step S109).

The center calculating section 137 sets the center 168 of the ellipse which is formed by the current elliptic data 166, as the center 168 of the blood vessel. The center 168 of the blood vessel is obtained for all the ultrasonic images which are read instep S106.

The center line calculating section 142 successively connects together the centers 168 of the blood vessel which are obtained by the center calculating section 137, to calculate the center line of the blood vessel (step S110).

FIG. 8 illustrates the calculation of the center line of the blood vessel. The left view of FIG. 8 is a view of the blood vessel as seen from the lateral side with respect to the longitudinal direction, and the right view of the figure is a view of the blood vessel as seen from the upper side with respect to the longitudinal direction.

When the center line calculating section 142 aligns the centers 168 of the vascular intima 164 along the coordinate axis of the reference coordinate system and in the longitudinal direction of the blood vessel, center lines 170a and 170b of the blood vessel such as shown in FIG. 8 are obtained.

The tomographic image producing section 144 rebuilds an image from the three-dimensional image so that the center line of the blood vessel which is calculated by the center line calculating section 142 is a straight line, and then extracts cross sections which extend along the center line to produce a pseudo long-axis tomographic image 180 of the blood vessel as shown in FIG. 9 (step S111). Even when the center line of the blood vessel which is actually measured is curved, the center line of the pseudo long-axis tomographic image 180 of the blood vessel produced by the tomographic image producing section 144 has a linear shape.

FIG. 9 illustrates production of the pseudo long-axis tomographic image. The tomographic image producing section 144 rebuilds an image so that the center line 170a of the blood vessel that is calculated by the center line calculating section 142 by connecting together the centers 168 of the ultrasonic images in which the centers 168 (see FIG. 7) are obtained in the process of step S109 is a straight line, and extracts cross sections which extend along the center line. As a result, as shown in FIG. 9, the contour of the vascular intima 164 is obtained. Therefore, the pseudo long-axis tomographic image 180 becomes an image in which the blood vessel is cut while passing through the center of the blood vessel from a direction parallel to the longitudinal direction of the blood vessel.

The blood vessel diameter calculating section 146 calculates the blood vessel diameter by using the pseudo long-axis tomographic image 180 produced by the tomographic image producing section 144 (step S112), and ends the operation of calculating the blood vessel diameter. The calculation of the blood vessel diameter is performed by calculating the distance from the center line 170a of the blood vessel to the vascular intima 164 obtained from the pseudo long-axis tomographic image 180. Specifically, the blood vessel diameter is obtained by adding together the length of a straight line which is dropped, perpendicularly to the center line 170a of the blood vessel, to the upper vascular intima 164, and that of a straight line which is dropped to the lower vascular intima 164.

Next, a method of applying the technique for calculating the blood vessel diameter as described above to an FMD test will be described. In an FMD test, as described above, avascularization is performed on the blood vessel for a constant period of time, and the vascular endothelial function is evaluated based on comparison between the blood vessel diameter at rest or before avascularization, and the blood vessel diameter when the vessel is maximally expanded after release of avascularization. Therefore, acquisition of a plurality of ultrasonic images which are necessary for calculating the blood vessel diameter is conducted one time at rest or immediately before avascularization, and continuously plural times after release of avascularization.

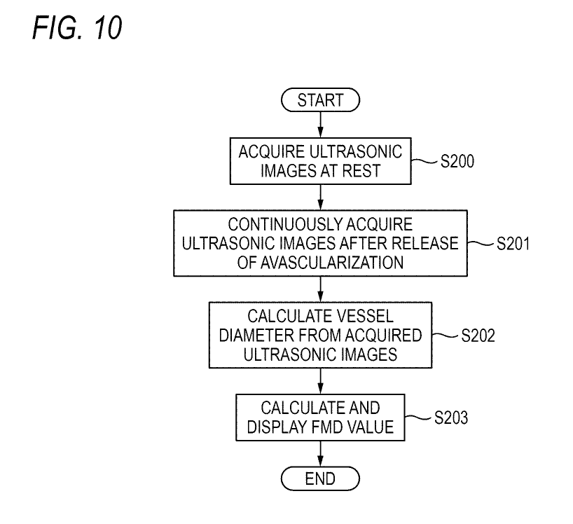

The operation will be specifically described with reference to the operation flowcharts of FIGS. 5, 6, and 10. FIG. 10 is an operation flowchart of a process in which the blood vessel diameter measured by the blood vessel diameter measuring apparatus of the embodiment is applied to an FMD test. First, ultrasonic images at rest are acquired (step S200). In this process, steps S100 to S105 in FIG. 5 are executed to acquire a plurality of ultrasonic images which are necessary for calculating the blood vessel diameter. When all necessary ultrasonic images have been acquired (step S105: YES), the operation of acquiring ultrasonic images is ended.

When the acquisition of ultrasonic images at rest is completed, the front arm is pressurized by the cuff to perform avascularization, while the ultrasonic probe 110 remains to be disposed above the blood vessel. After avascularization is performed for a constant period of time (for example, about five minutes), the avascularization is released, the operation of acquiring a plurality of ultrasonic images which are necessary for calculation of the blood vessel diameter is immediately started, and ultrasonic images after release of avascularization are continuously acquired (step S201). As described above, the operation of acquiring ultrasonic images after release of avascularization are continuously performed plural times in order to acquire the blood vessel diameter when the vessel is maximally expanded. Specifically, steps S101 to S105 in FIG. 5 are executed, and, when acquisition of ail ultrasonic images which are necessary for calculating the blood vessel diameter at timing t1 is ended (step S105; YES), one operation of acquiring ultrasonic images is ended.

When acquisition of ultrasonic images at time t1 is ended, the operation of acquiring ultrasonic images at next timing t2 is immediately started, and all necessary ultrasonic images are acquired (steps S101 to S105). As described above, ultrasonic images are continuously acquired in such a manner that, when all necessary ultrasonic images at a certain timing are acquired, the operation of acquiring ultrasonic images at the next timing is immediately started.

When ail ultrasonic images necessary for an FMD test have been acquired in this way, the blood vessel diameter is then calculated by using the ultrasonic images. Here, the case where the blood vessel diameter is calculated subsequently to the acquisition of ultrasonic images is described. As described above, however, the calculation may be performed after elapse of a certain period of time, or performed by an external apparatus such as a personal computer.

When all ultrasonic images are acquired in step S201, ultrasonic images are read out in step S202 from the ultrasonic image storing section 133 in which ultrasonic images are stored, and the blood vessel diameter is calculated. As described above, the calculation is performed by executing steps S106 to S112 in FIG. 6.

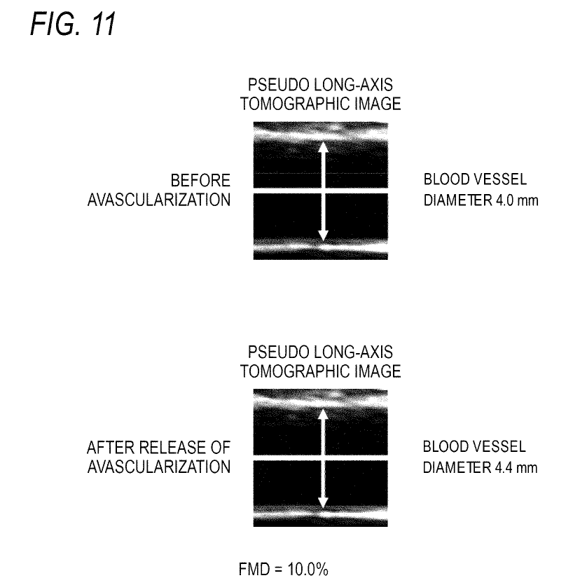

Next, the FMD value is calculated from the calculated blood vessel diameter before avascularization and the blood vessel diameter when the vessel is maximally expanded after release of avascularization, and the value is displayed on the displaying section 150 (step S203). FIG. 11 illustrates a display example of a result of the FMD test based on the blood vessel diameters before and after avascularization which are measured by the blood vessel diameter measuring apparatus of the embodiment. The term “display” includes a display on a flat display device such as a liquid crystal display (LCD), and printing on a sheet or the like.

In the display example, a situation where the blood vessel diameter before avascularization is 4.0 mm, that after release of avascularization is 4.4 mm, and the FMD value is 10.0% is displayed. The blood vessel diameter and FMD value displayed indicate that the vascular endothelial function of the subject is good.

In the above-described embodiment, a plurality of ultrasonic images of a blood vessel are acquired, the center of the blood vessel is collectively calculated with respect to the plurality of ultrasonic images, and then the center line of the blood vessel is calculated. In the blood vessel diameter measuring apparatus of the presently disclosed subject matter, alternatively, a process in which one ultrasonic image of a blood vessel is acquired, and the center of the blood vessel is calculated with respect to the one ultrasonic image may be repeated to calculate the center line of the blood vessel.

According to the blood vessel diameter measuring apparatus 100 of the presently disclosed subject matter, as described above, the ultrasonic probe 110 which is usually used is supported by the support fixture 120, and an ultrasonic image of a blood vessel is produced by using the ultrasonic signal of the ultrasonic probe 110 and the position/attitude information of the position/attitude detecting section 115. Therefore, the three-dimensional position of the ultrasonic probe 110 can be specified without using a dedicated ultrasonic probe. Consequently, a situation such as that where measured blood vessel diameters are different depending on the skill of the operator hardly occurs, and the blood vessel diameter can be accurately measured.

Moreover, ultrasonic images are made correspond to positions of the reference coordinate system by using the position/attitude information added to the ultrasonic images, and therefore ultrasonic images in various positions and attitudes can be aligned in the reference coordinate system.

The center of the extracted blood vessel is obtained by obtaining the center of an ellipse which is formed based on the shape of the vascular intima. Therefore, the center of the blood vessel can be correctly obtained.

Moreover, a pseudo long-axis tomographic image is produced by rebuilding ultrasonic images so that the center line of the blood vessel is a straight line, whereby the blood vessel diameter can be correctly obtained.

Furthermore, both blood vessel diameters before avascularization and when a blood vessel is maximally expanded after release of avascularization are obtained. When this process is applied to an FMD test, therefore, the state of the vascular endothelial function of the subject can be known, and it is possible to evaluate the degree of progression of arteriosclerosis.

What is claimed is:

1. A blood vessel diameter measuring apparatus comprising:
an ultrasonic probe which emits an ultrasonic wave toward a blood vessel, receives an ultrasonic wave reflected from the blood vessel and outputs an ultrasonic signal based on the received ultrasonic wave;
a position/attitude detecting section which is attached to the ultrasonic probe and outputs position/attitude information indicating a position of the ultrasonic probe and an attitude of the ultrasonic probe with respect to the blood vessel;
a support fixture which supports the ultrasonic probe so as to enable the ultrasonic probe to perform at least one of operations including:
inclination in a direction which is inclined with respect to a longitudinal direction of the blood vessel;
movement in the longitudinal direction of the blood vessel; and
rotation from the longitudinal direction of the blood vessel to a direction which intersects with the longitudinal direction;
an image processing section which produces an ultrasonic image of the blood vessel by using the ultrasonic signal output from the ultrasonic probe and the position/attitude information output from the position/attitude detecting section; and
a measuring section which measures a blood vessel diameter of the blood vessel by using the produced ultrasonic image.

2. The blood vessel diameter measuring apparatus according to claim 1, wherein the image processing section includes:
an ultrasonic image producing section which produces the ultrasonic image by using the ultrasonic signal, and which adds the position/attitude information to the produced ultrasonic image;
an ultrasonic image storing section which stores the produced ultrasonic image and the position/attitude information added to the ultrasonic image;
a coordinate converting section which, by using the position/attitude information added to the stored ultrasonic image, makes an altitude of the ultrasonic image correspond to a position of a reference coordinate system;
a vascular intima extracting section which extracts a vascular intima in a direction which is perpendicular to the longitudinal direction of the blood vessel, by using a coordinate-converted ultrasonic image; and
a center calculating section which obtains a center of the extracted blood vessel.

3. The blood vessel diameter measuring apparatus according to claim 2, wherein
the center calculating section calculates elliptic data based on a shape of the
extracted vascular intima, and obtains a center of an ellipse formed by the elliptic data, thereby obtaining the center of the extracted blood vessel.

4. The blood vessel diameter measuring apparatus according to claim 2, wherein the measuring section has:
a center line calculating section which successively connects the centers of the blood vessel to calculate a center line of the blood vessel;
a tomographic image producing section which rebuilds the ultrasonic image so that the calculated center line is a straight line, to produce a pseudo long-axis tomographic image; and
a blood vessel diameter calculating section which obtains the blood vessel diameter by using the produced pseudo long-axis tomographic image.

5. The blood vessel diameter measuring apparatus according to claim 1, wherein
the ultrasonic image is acquired in both modes or before avascularization of an artery or before a cuff is attached to a subject, and after release of avascularization of the artery.
